(19) United States
(12) Patent Application Publication
(10) Pub. No.: US 2025/0034508 A1
(43) Pub. Date: Jan. 30, 2025

(54) SYSTEMS FOR MEASURING A MICROORGANISM CONCENTRATION AND APPLYING A BIOCIDE, AND RELATED METHODS

(71) Applicant: Cameron International Corporation, Houston, TX (US)

(72) Inventors: James Fajt, College Station, TX (US); Michael Van Spankeren, Houston, TX (US)

(21) Appl. No.: 18/785,028

(22) Filed: Jul. 26, 2024

Related U.S. Application Data
(60) Provisional application No. 63/515,669, filed on Jul. 26, 2023.

(51) Int. Cl. C12M 1/34; C12M 1/00; C12M 1/36

(52) U.S. Cl. CPC C12M 41/36; C12M 29/14; C12M 41/32; C12M 41/48

(57) ABSTRACT

A system for measuring a microorganism concentration and applying a biocide includes an injection pump in fluid communication with at least one biocide tank and a fluid conduit in fluid communication with a wellbore, microorganism sensor in fluid communication with the fluid conduit and configured to determine a microorganism concentration of fluid in the fluid conduit, and a biocide controller in operable communication with the injection pump and the microorganism sensor. The biocide controller is configured to determine a biocide dosage to apply to the fluid based on the microorganism concentration, and control an operation of the injection pump to apply the biocide dosage to the fluid. Related methods and biocide application systems are also disclosed.

### CROSS REFERENCE TO RELATED APPLICATION

This application claims priority to and the benefit of U.S. Provisional Patent Application No. 63/515,669, filed on Jul. 26, 2023, entitled “AUTOMATIC BACTERIA MEASUREMENT SYSTEMS AND METHODS FOR OIL AND GAS BIOCIDE APPLICATION,” the disclosure of which is incorporated herein by reference in its entirety.

### FIELD

The present disclosure generally relates to biocides, for example as used in the oil and gas industry, and more particularly to use of automatic bacteria measurement systems and methods to control biocide application. In particular, the present disclosure relates to methods and systems for measuring a microorganism concentration in a fluid, determining a biocide dosage to apply to the fluid based on the measured microorganism concentration, and controlling operation of a biocide injection pump to provide the biocide dosage to the fluid.

### BACKGROUND

Many oilfield operations include the circulation of a fluid into and/or through a borehole or wellbore. Such fluids may include drilling fluids that are circulated during the drilling of the well, completion fluids that are circulated during or after drilling, and fracturing fluids which are used after drilling to stimulate the well to increase production from a hydrocarbon reservoir.

After a well is drilled into a subterranean earth formation that contains hydrocarbons (e.g., oil, natural gas) and/or water, one or more operations may be performed to increase the production of the hydrocarbons. To increase the permeability and flow of the formation fluids to the surface, the wells are often subjected to stimulation operations. Stimulation generally refers to several post drilling processes used to clean the wellbore, enlarge channels, and increase pore space of the earth formation in the region (zone) of the earth formation to be injected, increasing the permeability of that region of the earth formation.

Stimulation operations may include pumping stimulation fluids at high pressure and rate into the earth formation. The stimulation fluid may include water and high viscosity fluid additives. In some instances, the stimulation fluids include fracturing fluids including one or more proppants for maintaining the conductivity of fractures after the fluid pressure is released. The fracturing fluid may include a carrier fluid (usually water or a brine) and a polymer for reducing friction. The fracturing fluid may be provided into the earth formation at a pressure that exceeds the rock strength of the earth formation to open fractures in the earth formation. The fractures may extend from the wellbore into the earth formation as much as several hundred feet. The carrier fluid may include various additives, viscoelastic surfactant gels, gelled oils, crosslinkers, oxygen scavengers, and/or other additives.

While water is used in many wellbore fluids (e.g., drilling fluids, stimulation fluids, fracturing fluids, completion fluids, other fluids), the water may contain microbes, such as bacteria, fungus, etc., that can grow and proliferate on the surface of the wellbore, downhole, and/or on wellbore equipment. Biocides and antimicrobials may be used to inhibit, reduce, and/or remove microbial growth in the water. If left untreated, microbes and microbial biofilms (slimes) can cause deterioration of equipment, loss off efficiency in equipment, promotion and acceleration of corrosion on metal surfaces, or increased down time.

### BRIEF SUMMARY

In some configurations, a method for automatic biocide treatment includes automatically measuring bacteria population in a treatment target, and automatically determining a correct dose of an effective biocide based on the measured bacteria population.

The method can further include automatically applying the correct dose of the effective biocide to the treatment target. The treatment target can be repeatedly automatically measured at periodic time intervals. The treatment target can be an oil or gas reservoir.

In some embodiments, a system for measuring a microorganism concentration and applying a biocide includes an injection pump in fluid communication with at least one biocide tank and a fluid conduit in fluid communication with a wellbore, a microorganism sensor in fluid communication with the fluid conduit and configured to determine a microorganism concentration of fluid in the fluid conduit, and a biocide controller in operable communication with the injection pump and the microorganism sensor. The biocide controller includes at least one processor, and at least one non-transitory computer-readable storage medium having instructions thereon that, when executed by the at least one processor, cause the biocide controller to receive, from the microorganism sensor, the microorganism concentration of the fluid in the piping, based on the microorganism concentration of the fluid in the piping, determine a biocide dosage to apply to the fluid, and control an operation of the injection pump to apply the biocide dosage to the fluid.

In some embodiments, a method of autonomously treating a fluid with biocide includes measuring a microorganism concentration with a microorganism sensor in fluid communication with a fluid conduit configured to provide a fluid to a wellbore, receiving, with an analysis module, the microorganism concentration from the microorganism sensor, using the analysis module, determining a biocide dosage to apply to the fluid to reduce the microorganism concentration in the fluid based on the measured microorganism concentration in the fluid, and based on the determined biocide dosage, controlling an operation of an injection pump with a biocide application controller in operable communication with the analysis module.

In some embodiments, a biocide application system includes a microorganism sensor configured to determine a microorganism concentration within a fluid, and a biocide controller. The biocide controller includes at least one processor, and at least one non-transitory computer-readable storage medium having instructions thereon that, when executed by the at least one processor, causes the biocide controller to receive, from the microorganism sensor, the microorganism concentration, based on the microorganism concentration, determines at least one of a biocide flowrate, a biocide concentration, a biocide application duration, or a biocide application frequency to apply to the fluid, and causes a biocide injection pump to provide biocide to the fluid based on the at least one of the biocide flowrate, the biocide concentration, the biocide application duration, or the biocide application frequency.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a simplified schematic of a wellbore system including a biocide application system configured to control an amount of biocide to provide to one or more portions of the wellbore system, according to at least one embodiment of the present disclosure;

FIG. 2 is a simplified schematic of an environment in which the biocide application system may operate, according to at least one embodiment of the disclosure;

FIG. 3 is a simplified flow diagram illustrating a method of controlling biocide application, according to at least one embodiment of the disclosure; and

FIG. 4 illustrates certain components that may be included within a computer system, in accordance with at least one embodiment of the disclosure.

### DETAILED DESCRIPTION

In the following description, numerous details are set forth to provide an understanding of some embodiments of the present disclosure. It is to be understood that the following disclosure provides many different embodiments, or examples, for implementing different features of various embodiments. Specific examples of components and arrangements are described below to simplify the disclosure. These are, of course, merely examples and are not intended to be limiting. However, it will be understood by those of ordinary skill in the art that the system and/or methodology may be practiced without these details and that numerous variations or modifications from the described embodiments are possible. This description is not to be taken in a limiting sense, but rather made merely for the purpose of describing general principles of the implementations. The scope of the described implementations should be ascertained with reference to the issued claims.

As used herein, the terms “connect”, “connection”, “connected”, “in connection with”, and “connecting” are used to mean “in direct connection with” or “in connection with via one or more elements”; and the term “set” is used to mean “one element” or “more than one element”. Further, the terms “couple”, “coupling”, “coupled”, “coupled together”, and “coupled with” are used to mean “directly coupled together” or “coupled together via one or more elements”. As used herein, the terms “up” and “down”; “upper” and “lower”; “top” and “bottom”; and other like terms indicating relative positions to a given point or element are utilized to more clearly describe some elements. Commonly, these terms relate to a reference point at the surface from which drilling operations are initiated as being the top point and the total depth being the lowest point, wherein the well (e.g., wellbore, borehole) is vertical, horizontal or slanted relative to the surface.

The present disclosure generally relates to biocides, for example as used in the oil and gas industry, and more particularly to the use of automatic bacteria measurement systems and methods to control biocide application. The methods and systems for measuring and controlling biocide application may be employed in one or more downhole applications (e.g., in a wellbore), surface facilities (e.g., separators, tanks, pipelines, cooling water systems), transportation, storage, or in other applications that include the use and/or storage of water that may include one or more microorganisms. According to embodiments described herein, a microorganism concentration (e.g., also referred to as a “microbe concentration” or a “microbial concentration”) of a fluid may be measured in-situ and in real time without operator intervention. Based on the measured microorganism concentration, a biocide dosage may be calculated, and a biocide may be applied to the fluid based on the calculated biocide dosage in real time and without operator intervention (e.g., automatically). Accordingly, a microorganism concentration may be measured and, responsive to the measured microorganism concentration, a biocide may be applied to the fluid and/or to a system containing the fluid (e.g., a tank, a pipeline, equipment). The methods and systems may facilitate automatically measuring the microorganism concentration of a fluid, determining a biocide dosage to apply to the fluid based on the measured microorganism concentration, and automatically controlling the biocide dosage applied to the fluid. The microorganism concentration may include a concentration of one or more of (e.g., each of) different types of microorganisms (e.g., one or more of bacteria, fungi, algae, protozoans, spores from bacteria, fungal spores, pollen, fragments thereof, and/or sulfate-reducing microorganisms).

A biocide application system for measuring a microorganism concentration of a fluid and applying a biocide to the fluid includes a microorganism measurement manager configured to measure a concentration of a microbial population (e.g., a microorganism concentration, such as a bacteria concentration) in a fluid; and a biocide controller configured to apply a biocide to the fluid and reduce the concentration of the microbial population. The fluid may include water or a water-containing fluid. In some embodiments, the fluid is used in one or more wellbore operations, such as in one or more wellbore fluids. By way of non-limiting example, the fluid may include water, a water flooding fluid, a drilling fluid, a completion fluid, a stimulation fluid, a fracturing fluid, a hydrostatic testing fluid, a coiled tubing fluid, an intervention fluid, a cement slurry, a produced fluid (e.g., produced water), cooling water, a reservoir fluid from a reservoir located within an earth formation, or another fluid.

The microorganism measurement manager may include a microorganism sensor in fluid communication with the fluid. The microorganism sensor may be configured to receive and analyze a sample of the fluid to determine the microorganism concentration in the fluid. The fluid may include a fluid to be provided to a wellbore and/or an earth formation through which the wellbore extends. In some embodiments, the fluid includes a produced fluid, such as produced water. The microorganism sensor may be configured to measure (e.g., determine) a concentration of microorganisms (e.g., bacteria) in the fluid. The microorganism measurement manager may be configured to determine the microorganism concentration on-site at a location where the fluid (and the wellbore) is located. The microorganism measurement manager may be configured to determine the microorganism concentration in-situ, in real time, and without operator intervention. The microorganism measurement manager may be configured to determine the microorganism concentration at predetermined intervals, such as every month, every week, every day, two times a day, four times a day, six times a day, twelve times a day, or at other intervals.

The biocide controller may be in operable communication with the microorganism measurement manager. In some embodiments, the biocide controller includes an analysis module (e.g., biocide calculator) configured to determine one or both of a type of biocide (e.g., a composition of biocide) to apply to the fluid or to a system containing the fluid, or a biocide dosage based, at least in part, on the microorganism concentration. The analysis module may be configured to receive an output from the microorganism measurement manager, the output indicative of the microorganism concentration in the fluid. Responsive to receiving the microorganism concentration from the microorganism measurement manager, the analysis module determines one or more of a type of biocide to apply or a biocide dosage to apply to the fluid, a system including the fluid, and/or a system configured to be in fluid communication with the fluid. The biocide dosage may include one or more of a flowrate of the biocide, a concentration of the biocide, a frequency of application of the biocide, or a duration over which to apply the biocide. In some embodiments, in addition to the microorganism concentration, the analysis module may receive one or more of properties (e.g., a temperature, pressure, pH, salinity, ion concentration, composition) of the fluid, a property (e.g., temperature, pressure) of an asset (e.g., wellbore equipment) to be protected by the biocide, a cost of the biocide, or a budget for the biocide. In some embodiments, the analysis module determines the type of biocide and/or the biocide dosage based, at least in part, on the property of the fluid, the property of the asset, the cost of the biocide, or the budget for the biocide.

The biocide controller may further include a biocide application controller. The biocide application controller may receive one or more of the determined type of biocide to apply or the biocide dosage from the analysis module. Responsive to receiving the type of biocide to apply or the biocide dosage, the biocide application controller is configured to control operation of an injection pump configured to provide the biocide to the fluid. For example, the biocide application controller may control one or more of a pump speed of the injection pump, a frequency of operation of the injection pump, power to the injection pump (e.g., an on and off status of the injection pump), or another condition of the injection pump. In some embodiments, the biocide application controller is in operable communication with one or more valves configured to fluidly couple different biocide compositions to the injection pump. The biocide application controller may be configured to cause the valves to open and close to a desired position (e.g., fully open, fully closed, percentage of fully open) to provide a desired biocide composition and flowrate of the biocide to the injection pump and the fluid.

Accordingly, the biocide application system including the microorganism measurement manager and the biocide controller facilitates the autonomously treating and automatic measurement of the microorganism concentration of a fluid and application of biocide to the fluid to reduce and/or control the concentration of the biocide in the fluid, the wellbore, and the earth formation. For example, the microorganism measurement manager may facilitate the continuous measurement of the microorganism concentration in the fluid at predetermined intervals (and without operator intervention, such as without an operator physically collecting a sample of the fluid and analyzing the fluid in a laboratory or with a handheld device). The biocide controller may receive the microorganism concentration and, based at least in part on the microorganism concentration, determine a biocide and a biocide dosage to apply to the fluid and/or a system associated with the fluid to reduce and/or control the concentration of the biocide to a desirable level. Responsive to determining the biocide dosage, the biocide controller may cause an injection pump and/or one or more valves in fluid communication with a biocide to provide the biocide at the biocide dosage to the fluid. The biocide controller may determine the biocide dosage and cause the injection pump to provide the biocide dosage to the fluid autonomously (e.g., autonomously treating), in-situ, in real time, and without operator intervention.

By way of comparison, conventional methods of controlling biocide application involve physically sampling a fluid to measure a bacteria concentration and then adjusting an application rate of the biocide based on the bacteria concentration. However, wellbores and fluids provided to the wellbore may be located at remote locations, making it difficult for an operator to sample the fluid and adjust application of the biocide. Accordingly, the bacteria concentrations are measured infrequently, such as fewer than quarterly or even fewer than annually. The infrequent measurement of bacteria concentration results in overapplication or underapplication of the biocide, resulting in increased biocide costs and/or operating costs of the wellbore.

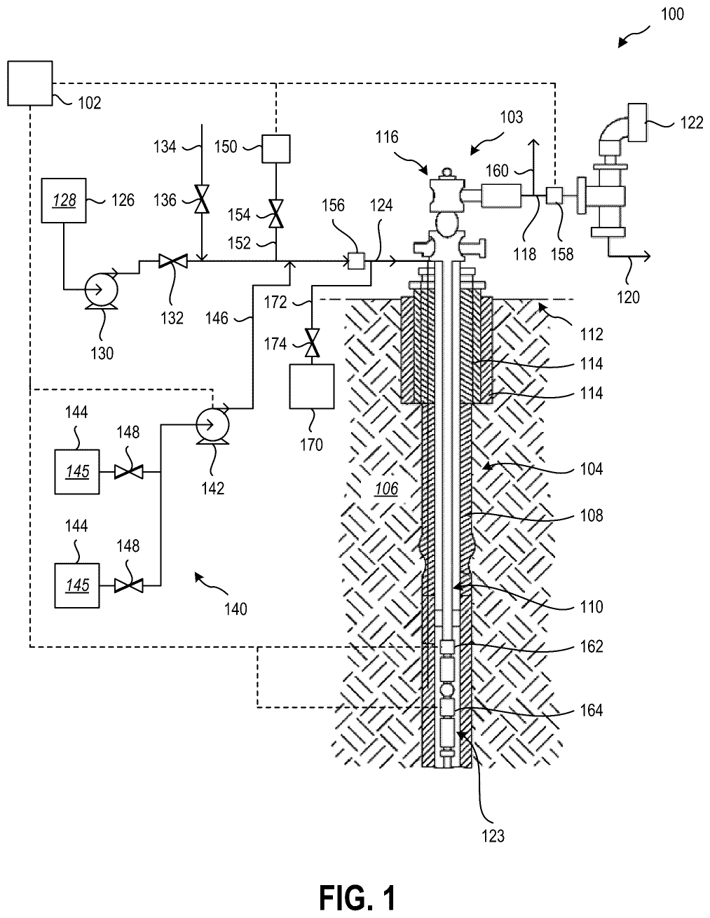

FIG. 1 is a simplified schematic of a wellbore system 100 including a biocide application system 102 (also referred to as a “biocide application manager”) configured to autonomously (and automatically) measure a microorganism concentration and control an amount of biocide to provide to one or more portions of the wellbore system 100 based on the microorganism concentration, according to at least one embodiment of the present disclosure. As described herein, the biocide application system 102 may be configured to measure a microorganism concentration of one or more wellbore fluids and, responsive to the measured microorganism concentration, determine a biocide dosage to apply to the wellbore fluid and/or to at least a portion of the wellbore system 100. The biocide dosage may include one or more of (e.g., each of) a concentration of biocide, flowrate (e.g., an application rate) of biocide, a duration of application of biocide, or a frequency of biocide application. The biocide dosage may include each of the concentration of biocide, the flowrate of biocide, a duration of application of biocide, and a frequency of biocide application. Responsive to determining the biocide dosage, the biocide application system 102 may be configured to control application of biocide to one or more components of the wellbore system 100, such as injection of the biocide into the wellbore 104. In some embodiments, the biocide application system is configured to determine a type of biocide to apply and/or a composition of biocide to apply based on the measured microorganism concentration.

The wellbore system 100 may include a well 103 having a wellbore 104 extending into (through) an earth formation 106. The wellbore 104 may extend through one or more reservoirs (e.g., hydrocarbon-containing reservoirs, water reservoirs) located within the earth formation 106. For example, the wellbore 104 may extend through one or more hydrocarbon-containing zones of the earth formation 106. The wellbore 104 may include, for example, tubing 108 extending into the earth formation 106 and configured to provide one or more chemicals (e.g., biocide) to the wellbore 104. The wellbore 104 may further include production tubing 110 configured to provide one or more produced fluids to a surface 112. In some embodiments, the wellbore 104 includes one or more sections of cement 114 between the tubing 108 and surfaces of the earth formation 106.

At the surface 112, the wellbore system 100 includes a wellhead 116 including a so-called “Christmas tree” including piping and connections for operating the well 103. The production tubing 110 may be in fluid communication with the wellhead 116 and configured to provide produced fluids (e.g., oil, gas, water) to piping 118 in fluid communication with surface piping 120. A choke 122 may be configured to control the pressure of the wellbore 104 and the flow of the produced fluids to the surface piping 120. An electric submersible pump 123 may be located within the wellbore 104 and configured to provide one or more chemicals to the wellbore 104 and/or to facilitate the flow of produced fluids to the surface piping 120.

With continued reference to FIG. 1, the wellbore system 100 may include a fluid conduit 124 configured to be in fluid communication with the wellbore 104, such as with the tubing 108. The fluid conduit 124 may be referred to as “piping,” “tubing,” or “wellbore fluid injection piping” and may be configured to provide one or more wellbore fluids to the wellbore 104. The fluid conduit 124 may be configured to provide a fluid to the wellbore 104. In some embodiments, the fluid conduit 124 is configured to provide a fluid to the earth formation 106, such as to hydrocarbon-containing reservoirs within the earth formation 106.

In some embodiments, the fluid conduit 124 is in fluid communication with, for example, a tank 126 including a water-containing fluid 128. The water-containing fluid 128 may be provided to the wellbore 104 and/or mixed with one or more other materials to form one or more wellbore fluids to provide to the wellbore 104. The one or more wellbore fluids may include one or more of a water flooding fluid, a drilling fluid, a completion fluid, a stimulation fluid, a fracturing fluid, A hydrostatic testing fluid, a coiled tubing fluid, an intervention fluid, a cement slurry, or another wellbore fluid. In some embodiments, a pump 130 is configured to provide a flowrate of the water-containing fluid 128 to the wellbore 104 via the fluid conduit 124. A valve 132 may be configured to control fluid communication between the fluid conduit 124 and the tank 126.

In some embodiments, wellbore fluid piping 134 is in fluid communication with one or more wellbore fluids and the fluid conduit 124. The wellbore fluid piping 134 may be configured to provide one or more wellbore fluids to the wellbore 104 via the fluid conduit 124. In some embodiments, the wellbore fluid from the wellbore fluid piping 134 is configured to be mixed with the water-containing fluid 128 in the fluid conduit 124. A valve 136 may facilitate controlling the flow of the wellbore fluid from the wellbore fluid piping 134 to the fluid conduit 124.

With continued reference to FIG. 1, the biocide application system 102 may include a biocide injection system 140 including an injection pump 142 (a biocide injection pump) in operable and fluid communication with one or more biocide tanks 144. The biocide tanks 144 may be in fluid communication with biocide injection piping 146 configured to provide biocide from one or more of the biocide tanks 144 to a fluid (e.g., a wellbore fluid) in the fluid conduit 124. The biocide tanks 144 may each individually include a biocide 145 therein. Although FIG. 1 illustrates that the biocide injection system 140 includes two biocide tanks 144, the disclosure is not so limited and the biocide injection system 140 may include one biocide tank 144, or more than two biocide tanks 144, such as three, four, five, or more biocide tanks 144. In some embodiments, each of the biocide tanks 144 includes a different biocide therein. The biocide tanks 144 may individually be in operable communication with the injection pump 142 and the with biocide injection piping 146 via valves 148.

The biocide 145 may include one or more materials formulated and configured to reduce a microorganism concentration of the fluid provided to the wellbore 104, fluids within the wellbore 104, and/or produced fluids within the wellbore 104. In some embodiments, the biocide is configured to reduce a microorganism concentration of the fluid in the fluid conduit 124. The biocide 145 may be formulated and configured to be compatible with the wellbore system 100, such as with the wellbore fluid. The biocide 145 may include one or more of glutaraldehyde, tetrakis-hydroxymethyl-phosphonium sulfate (THPS), quaternary ammonium compounds (quats), sodium hypochlorite, chlorine dioxide, hydrogen peroxide, methyl isothiocyanate (MITC), triazine (e.g., hexahydro-1,3,5-tris-(-2-hydroxyethyl)-s-triazine), ozone, bromous acid, perchloric acid, bronopol (e.g., 2-bromo-2-nitropropane-1,3-diol), isothiazoline(s) (e.g., alkyl isothiazonlin-3-ones), tributyl tetradecyl phosphonium chloride (TTPC), another material, or blends thereof. However, the disclosure is not so limited, and the biocide may include other materials.

The biocide injection piping 146 may be in fluid communication with the fluid conduit 124 and configured to provide biocide to the fluid conduit 124. In some embodiments, the biocide injection piping 146 is configured to facilitate provision of biocide from one or more of the biocide tanks 144 to the fluid conduit 124 between the wellbore 104 and each of the tank 126 and the wellbore fluid piping 134. In other words, the biocide injection piping 146 may be in fluid communication with the fluid conduit 124 downstream of the tank 126 and the wellbore fluid piping 134. While FIG. 1 illustrates that the biocide injection piping 146 at a particular location within the wellbore system 100, the disclosure is not so limited. In some embodiments, the biocide injection piping 146 is located at any location configured to provide the biocide 145 to the wellbore 104 and/or a produced fluid. For example, the biocide injection piping 146 may configured to be provided to the wellbore 104 or a produced fluid through one or more connections at the wellhead 116. In some embodiments, the biocide injection piping 146 is in fluid communication with the piping 118 and/or the surface piping 120 to provide the biocide 145 to, for example, a produced fluid.

The fluid conduit 124 may include one or more sensors configured to measure one or more properties of the fluid (e.g., the wellbore fluid) in the fluid conduit 124. For example, a microorganism sensor 150 may be located within and/or in fluid communication with the fluid conduit 124. In some embodiments, the microorganism sensor 150 is in fluid communication with the fluid conduit 124 such as through a sample line 152. The sample line 152 may be configured to collect (e.g., receive) a sample of the fluid from the fluid conduit 124 and provide the sample to the microorganism sensor 150. In some embodiments, the sample line 152 includes a valve 154 between the microorganism sensor 150 and the fluid conduit 124.

The microorganism sensor 150 (e.g., and the sample line 152) may be between the wellbore 104 and each of the tank 126 and the wellbore fluid piping 134. In some embodiments, the microorganism sensor 150 is between the wellbore 104 and the biocide injection piping 146. Accordingly, the microorganism sensor 150 may be located downstream of the biocide injection piping 146. In some embodiments, the microorganism sensor 150 may be configured to measure one or more properties of the fluid in the fluid conduit 124 after introduction of biocide or other materials or additives into the fluid such that the microorganism sensor 150 is configured to determine an effectiveness of biocide application to the fluid after application of the biocide begins or changes. The location of the microorganism sensor 150 downstream of the biocide injection piping 146 may facilitate determining an effectiveness and/or an effect of changing one or more application parameters of the biocide to the fluid in the fluid conduit 124.

In some embodiments, the microorganism sensor 150 is between biocide injection piping 146 and each of the tank 126 and the wellbore fluid piping 134. In other words, the microorganism sensor 150 may be located upstream of the biocide injection piping 146. In some such embodiments, the microorganism sensor 150 may be configured to measure one or more properties of the fluid in the fluid conduit 124 prior to introduction of biocide or other materials or additives into the fluid. In other words, the microorganism sensor 150 may measure the microorganism concentration of the fluid and the biocide injection system 140 may apply the biocide 145 to the fluid downstream of the microorganism sensor responsive to the measured microorganism concentration. In some embodiments, the biocide control system includes the microorganism sensor 150 upstream of the biocide injection piping 146 and an additional microorganism sensor 170 downstream of the biocide injection piping 146. The additional microorganism sensor 170 may be located downstream of the biocide injection piping 146 and the microorganism sensor 150. The additional microorganism sensor 170 may be configured to measure the microorganism concentration of the treated fluid downstream of the biocide injection piping 146. In some embodiments, the additional microorganism sensor 170 is in fluid communication with the fluid conduit 124 downstream of the biocide injection piping 146 via a sample line 172. A valve 174 may be configured to isolate the additional microorganism sensor 170 from the fluid conduit 124. In some embodiments, the additional microorganism sensor 170 is configured to measure a microorganism concentration of the treated fluid in the fluid conduit 124 to determine the effectiveness of biocide application. A difference between the microorganism concentrations measured by the microorganism sensor 150 upstream of the biocide injection piping 146 and the microorganism sensor 150 downstream of the biocide injection piping 146 may be an indication of the effectiveness of the biocide application to the fluid.

The microorganism sensor 150 may be configured to measure a microorganism concentration within the fluid in the fluid conduit 124. In some embodiments, the microorganism sensor 150 is configured to determine an enzyme activity level per until volume (e.g., per milliliter (mL)). In some embodiments, the microorganism sensor 150 is configured to determine the concentration of the microorganisms within the fluid as a number of cells of microorganisms per unit volume of the fluid (cells/mL). In some embodiments, the microorganism sensor 150 is configured to determine the concentration of the microorganisms within the fluid as colony-forming units (CFU) per unit volume of the fluid (e.g., CFU/mL). Of course, the description is not so limited, and the microorganism sensor 150 may be configured to determine the concentration of the microorganisms within the fluid as other units. In some embodiments, the microorganism sensor 150 is configured to determine one or more properties of the fluid (e.g., a fluorescence) which may correlate to the concentration of the microorganisms in the fluid.

As described above, the microorganism sensor 150 may be configured to measure the concentration of microorganisms in the fluid within the fluid conduit 124. The microorganism sensor 150 may include an automated device configured to collect a sample of the fluid from the fluid conduit 124 in-situ without operator intervention and in real time. The microorganism sensor 150 may include a self-contained unit configured to autonomously determine the concentration of microorganisms in the fluid. In some embodiments, the microorganism sensor 150 is configured to determine total living planktonic bacteria in the fluid. By way of non-limiting example, the microorganism sensor 150 may be configured to collect a sample of the fluid from the sample line 152. The fluid is filtered in the microorganism sensor 150 to capture planktonic bacteria. The filter may include, for example, a 0.2 μm ceramic filter. The filter may be configured to filter microorganisms from the sample such that the sample passes through and the microorganisms (planktonic bacteria) are collected on the filter. After filtering the microorganisms, the filter is contacted with (e.g., saturated with) an analysis chemistry, which may include a fluorescently-labelled enzyme substrate dye complex. The fluorescent-labelled enzyme substrate may react with an enzyme activity in living microorganisms (bacteria). Thus, if the microorganisms on the filter are living, a fluorescent compound (e.g., a phosphorescent dye) may be released responsive to contacting the microorganisms with the fluorescently labelled enzyme substrate dye complex. The dye may be extracted from the filter by rinsing the filter into a container, followed by measuring the fluorescence of the extracted dye with an optical device (e.g., a fluorometer). In some embodiments, the measured fluorescence is adjusted for temperature, reaction time, and sample volume, according to an algorithm used as a proxy for the total living microorganism concentration per volume (mL) of sample. The fluorescence of the extracted dye may be proportional to the microorganism concentration. Based on the sample volume and the measured fluorescence, the microorganism concentration may be calculated.

The microorganism sensor 150 may be configured to measure a concentration of microorganisms in the fluid within the fluid conduit 124 substantially continuously. In some embodiments, the microorganism sensor 150 is configured to measure the microorganism concentration at a predetermined schedule (e.g., a predetermined frequency). For example, the microorganism sensor 150 may determine the microorganism concentration monthly, two times per month, four times per month, weekly, two times per week, four times per week, daily, two times per day, four times per day, six times per day, every two hours, every hour, or at other durations. The microorganism sensor 150 may be configured to measure the concentration of microorganisms at least monthly, two times per month, four times per month, weekly, two times per week, four times per week, daily, two times per day, four times per day, six times per day, every two hours, every hour, or other durations.

After determining the microorganism concentration, the microorganism sensor 150 may be cleaned, such as by an automated cleaning in place (CIP) procedure wherein the filter is flushed with a solution (e.g., water, a cleaning solution) to remove any remaining dye and filtered microorganisms from the filter.

The microorganism sensor 150 may include a sensor commercially available from BactiQuant of Birkerod, Denmark. In some embodiments, the microorganism sensor 150 is configured to determine the microorganism concentration using a test described in U.S. Pat. No. 7,939,285, issued May 10, 2011, the disclosure of which is hereby incorporated herein in its entirety. However, the disclosure is not so limited, and the microorganism sensor 150 may determine the microorganism concentration by other methods. In some embodiments, the microorganism sensor 150 is configured to determine the microorganism concentration as described in U.S. Pat. No. 9,732,372, issued Aug. 15, 2017, the disclosure of which is hereby incorporated herein in its entirety.

With continued reference to FIG. 1, the wellbore system 100 may include one or more sensors configured to measure one or more properties of the fluids within the wellbore system 100. For example, a sensor 156 may be located within the fluid conduit 124 and configured to measure one or more properties of the fluid within the fluid conduit 124. In some embodiments, the sensor 156 is located proximate the microorganism sensor 150. In some embodiments, the sensor 156 is located between the microorganism sensor 150 and the wellbore 104. For example, the sensor 156 may be located downstream of the microorganism sensor 150. The sensor 156 may be configured to measure one or more of a temperature, a pressure, a salinity, a pH, or another property of the fluid in the fluid conduit 124.

The wellbore system 100 may further include a sensor 158 in the piping 118 and/or the surface piping 120. The sensor 158 may be configured to measure one or more conditions of the fluid in the piping 118 and/or the surface piping 120. For example, the sensor 158 may be configured to measure one or more of a temperature, a pressure, a salinity, a pH, or another property of the fluid in the piping 118 and/or the surface piping 120.

A sensor 162 may be located downhole within the wellbore 104 and configured to measure one or more conditions of a fluid within the wellbore. The one or more conditions may include one or more of a temperature, a pressure, a salinity, a pH, a concentration of one or more ionic species, or another property of the fluid within the wellbore 104.

In some embodiments, the wellbore system 100 includes a sensor 164 configured to measure one or more conditions of equipment and/or piping within the wellbore 104. The sensor 164 may be configured to measure one or more of a temperature, a pressure, or another condition of equipment and/or piping within the wellbore 104.

The biocide application system 102 may be in operable communication with each of the microorganism sensor 150, the injection pump 142, and the sensors 156, 158, 162, 164, as shown by dashed lines. As described herein, the biocide application system 102 may be configured to control one or more operating conditions of the injection pump 142 and/or the valves 148 based on the output of the microorganism sensor 150 (e.g., the microorganism concentration measured by the microorganism sensor 150).

While the wellbore system 100 has been described and illustrated as including the microorganism sensor 150 in fluid communication with the fluid conduit 124 providing a fluid into the wellbore 104, in some embodiments, the wellbore system 100 includes a microorganism sensor 150 in fluid communication with the piping 118 and/or the surface piping 120 and configured to measure a microorganism concentration of a produced fluid, such as produced water. For example, the microorganism sensor 150 may be configured to be in fluid communication with the produced fluid via a produced fluid sample line 160.

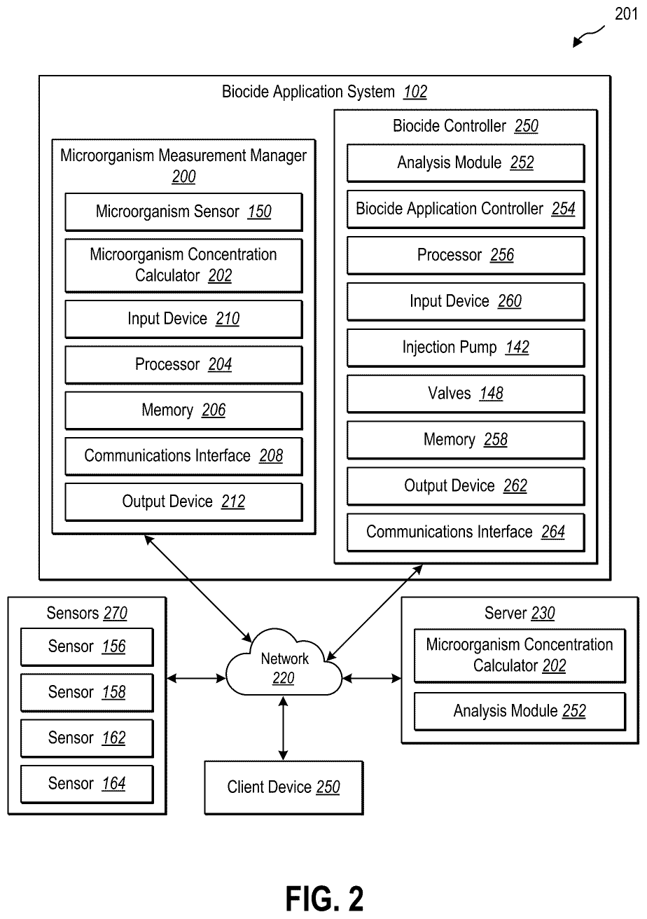

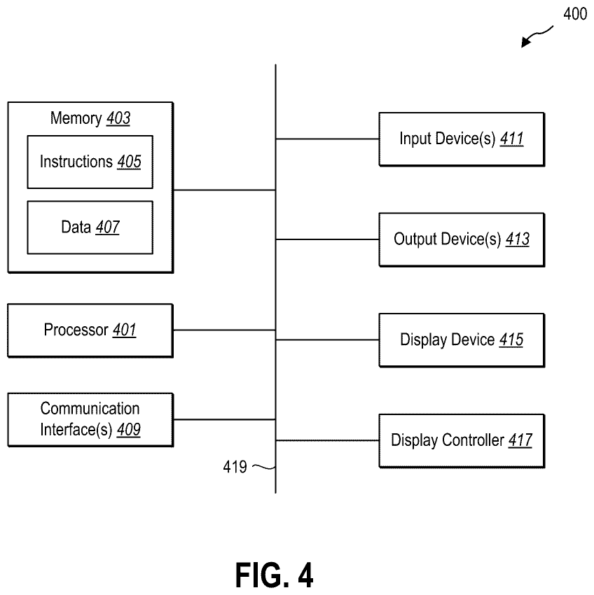

FIG. 2 is a simplified schematic of an environment 201 in which the biocide application system 102 may operate, according to at least one embodiment of the disclosure. The environment 201 may include a biocide manager including the biocide application system 102. FIG. 2 illustrates the biocide application system 102 in more detail than that described and illustrated in FIG. 1. The biocide application system 102 may include a microorganism measurement manager 200 and a biocide controller 250. The biocide manager further includes a network 220 configured to facilitate operable communication between one or more of the microorganism measurement manager 200, the biocide controller 250, a server 230, and/or a client device 240. In some embodiments, the environment 201 further includes sensors 270 configured to determine one or more properties of the one or more components of the wellbore system 100. The biocide controller 250 may be in operable communication with the microorganism measurement manager 200, such as by wired or wireless communication. In some embodiments, the biocide controller 250 is in operable communication with the microorganism measurement manager 200 via the network 220. In some embodiments, the biocide application system 102 may take the form of an IoT (Internet of Things) device configured to integrate and process equipment data at the edge. It will be appreciated that each of the various computing devices shown in FIG. 2 (e.g., the server 230, client device(s) 240, the microorganism measurement manager 200, the biocide controller 250) may refer to a variety of computing devices and may serve a particular embodiment of the biocide manager of the environment 201. Each of these devices may share some or all of the features discussed below in connection with FIG. 4.

The microorganism measurement manager 200 includes the microorganism sensor 150, a microorganism concentration calculator 202 configured to determine a concentration of microorganisms in a fluid (e.g., the wellbore fluid), a processor 204, memory 206, a communications interface 208, an input device 210, and an output device 212. The microorganism concentration calculator 202 may include software (e.g., one or more algorithms) configured of determine the microorganism concentration of a fluid based on an output from the microorganism sensor 150. The microorganism concentration calculator 202 may be configured to correct and/or adjust the determined microorganism concentration for temperature, sample time, and sample volume. In some embodiments, the microorganism measurement manager 200 may refer to any processing unit (e.g., microprocessor, computer processing unit (CPU), programmable hardware, edge of things (EoT) processor) that can be programmed or otherwise configured to receive an output (e.g., sensor data) from the microorganism sensor 150 and analyze the output to determine, for example, a concentration of microorganisms.

The processor 204 may be in electronic communication with the memory 206. The memory 206 may be any electronic component capable of storing electronic information. For example, the memory 206 may be embodied as random-access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof. Instructions and data may be stored in the memory 206. The instructions may be executable by the processor 204 to implement some or all of the functionality disclosed herein, such as to determine the microorganism concentration based on the output of the microorganism sensor 150.

The communications interface 208 may be configured to facilitate communication between the microorganism measurement manager 200 and one or more other electronic devices. The communication interface(s) 208 may be based on wired communication technology, wireless communication technology, or both. Some examples of communication interfaces 208 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

The input device 210 may include one or more of a keyboard, mouse, microphone, remote control device, button, joystick, trackball, touchpad, and lightpen. The output device 212 may include, for example, a display device, such as a screen or other display.

In some embodiments, each of the microorganism concentration calculator 202, the processor 204, the memory 206, the communications interface 208, the input device 210, and the output device 212 are part of the microorganism sensor 150 and the microorganism sensor 150 is configured to directly output the microorganism concentration.

The biocide controller 250 may include an analysis module 252 (also referred to as a “biocide determination and application calculator”) configured to determine a type of biocide and/or a biocide dosage to apply to a fluid, a biocide application controller 254, the injection pump 142, the valves 148, a processor 256, memory 258, an input device 260, an output device 262, and a communications interface 264. The input device 260, the output device 262, and the communications interface 264 may be substantially the same as the input device 210, the output device 212, and the communications interface 208. The analysis module 252 may be configured to facilitate determining a type of biocide to apply and/or a biocide dosage (e.g., flowrate, duration, frequency, concentration). For example, the memory 258 may include instructions thereon, that when executed by the processor 256, cause the analysis module 252 to determine a type of biocide to apply and/or a biocide dosage to apply. The

As described above, the biocide controller 250 may be in operable communication with the microorganism measurement manager 200. In some embodiments, the biocide controller 250 is configured to receive, from the microorganism measurement manager 200 (e.g., either directly, or through, for example, the network 220), the microorganism concentration or one or more parameters indicative of the microorganism concentration measured by the microorganism sensor 150 and/or calculated by the microorganism concentration calculator 202. The microorganism concentration or one or more parameters indicative of the microorganism concentration measured by the microorganism sensor 150 and/or calculated by the microorganism concentration calculator 202 may be referred to herein as “microorganism data.”

In some embodiments, the biocide controller 250 may refer to any processing unit (e.g., microprocessor, computer processing unit, programmable hardware, edge of things processor) that can be programmed or otherwise configured to receive an output (e.g., microorganism data) from the microorganism measurement manager 200 and analyze the output to determine, for example, a type of biocide (e.g., a composition of biocide) to apply and/or a biocide dosage for application of the biocide. The biocide dosage may include one or more of (e.g., each of) a biocide concentration, a biocide flowrate, the duration of application of biocide, and/or a frequency of application of the biocide.

Responsive to receiving the microorganism concentration from the microorganism measurement manager 200, the analysis module 252 may be configured to determine one or more of (e.g., each of) a type of biocide to apply to the fluid conduit 124 (FIG. 1) and at least one biocide dosage. In embodiments where the wellbore system 100 includes only one type of biocide (e.g., one biocide tank 144 including one biocide 145), the analysis module 252 may include a biocide application calculator configured to determine at least one biocide application parameter.

The analysis module 252 may determine the type of biocide and the at least biocide dosage, at least in part, on the microorganism data received from the microorganism measurement manager 200. By way of non-limiting example, the analysis module 252 may determine the type of biocide, the biocide dosage, or both based, at least in part, on one or more of (e.g., any of, one of, each of) compatibility of the biocide with the wellbore 104, wellbore fluids, wellbore equipment, and/or other piping and equipment to be contacted by the biocide, the type of microorganisms (e.g., the type of bacteria) to be treated by the biocide, the application of a corrosion inhibitor (e.g., whether a corrosion inhibitor is applied, the application rate of the corrosion inhibitor(s)), properties and conditions of the fluid to be treated (e.g., one or more of the temperature, the pH, and the composition of the fluid to be treated), the destination of the treated fluid, environmental regulations, retention time of the treated fluid, concentration of the biocide, availability of the biocide, and other factors determined by a customer and/or applicator of the biocide. In some embodiments, the analysis module 252 determines the concentration of the biocide to apply based on a recommended dosage provided by the supplier of the biocide, which may be provided (e.g., in the form of a table) in the memory 258. In some embodiments, the analysis module 252 determines the concentration of the biocide to apply using a data stored in memory 258, such as a look-up table.

In some embodiments, the analysis module 252 determines the type of biocide and the at least one biocide dosage, at least in part, on data stored in the memory 258. By way of non-limiting example, the memory 258 may include data indicative of one or more components of the wellbore system 100 (e.g., the types and/or costs of downhole equipment (e.g., pumps, valves, components of a bottomhole assembly), asset types intended to be protected by the biocide (e.g., equipment of the wellbore system 100), and/or the cost of various biocides (e.g., dollars per gallon). In some embodiments, the data is received from the input devices 260. The analysis module 252 may determine the type of biocide and/or the biocide dosage based, at least in part, on the data stored in the memory 258.

By way of non-limiting example, in some embodiments, where the cost of the biocide application (e.g., as determined by a concentration, a duration, and frequency of biocide application) exceeds the cost of assets (e.g., equipment) to be protected by the biocide, the analysis module 252 may determine not to apply the biocide, may determine to apply a less expensive biocide, and/or may determine to lower a dosage rate (e.g., one or more of a concentration, a duration, or a frequency) of the biocide.

The biocide application controller 254 may be in operable communication with the analysis module 252. The biocide application controller 254 may be configured to control one or more operations of the injection pump 142 and/or the valves 148 based, at least in part, on data received from the analysis module 252 (e.g., based on the determined biocide type and/or biocide dosage received from the analysis module 252). For example, the analysis module 252 may provide one or both of the type of biocide to apply and at least one biocide dosage to the biocide application controller 254. The biocide application controller 254 may be in operable communication with the injection pump 142 and the valves 148. Responsive to receiving the type of biocide to apply and/or the at least one biocide dosage, the biocide application controller 254 may control at least one operating condition of one or more of the injection pump 142 and the valves 148 based, at least in part, on one or both of the type of biocide to apply and/or the at least one biocide dosage determined by the analysis module 252. In some embodiments, the biocide application controller 254 may control one or more of a speed of the injection pump 142, a frequency of operation of the injection pump 142, or a duration of operation of the injection pump 142.

As a non-limiting example, the analysis module 252 may determine a particular type of biocide to apply, such as a particular composition of biocide to apply or mixture of biocides to apply. Responsive to receiving the data from the analysis module 252, the biocide application controller 254 may cause the valve 148 in fluid communication with the selected biocide tank 144 to open, and cause valves 148 in fluid communication with other biocide tanks 144 to close. As another non-limiting example, the analysis module 252 may determine at least one biocide dosage and, responsive to receiving the at least one biocide dosage from the analysis module 252, the biocide application controller 254 may control one or more operating conditions of the injection pump 142 and/or the valves 148 to control the at least one biocide dosage. In some embodiments, the biocide application controller 254 causes the injection pump 142 to operate at a speed to provide the biocide at a desired flow rate. In some embodiments, the biocide application controller 254 causes the injection pump 142 and the valve 148 to provide the biocide at the desired flow rate. In some embodiments, the biocide application controller 254 causes the injection pump 142 to operate at a desired frequency and/or for a desired duration. Accordingly, the biocide application controller 254 may control operation of the injection pump 142 and/or the valves 148 based on the determined biocide to apply and/or the biocide dosage.

With continued reference to FIG. 2, the environment may further include the sensors 270. The sensors 270 may include, for example, sensors 156 configured to determine one or more properties of a fluid in the fluid conduit 124, sensors 158 configured to determine one or more properties of a produced fluid, sensors 162 configured to determine one or more properties within the wellbore 104, and sensors 164 configured to determine one or more properties of wellbore equipment.

In some embodiments, the analysis module 252 is further configured to determine the type of biocide and the at least one biocide dosage based, at least in part, on data from the sensors 270. For example, the analysis module 252 may be configured to determine a type of biocide and/or a biocide dosage based on one or more properties of the fluid in the fluid conduit 124. In some embodiments, the analysis module 252 is configured to determine a type of biocide and/or a biocide dosage based on one or more properties of the produced fluid (e.g., in the piping 118). In some embodiments, the analysis module 252 is configured to determine a type of biocide and/or a biocide dosage based on one or more properties of a fluid within the wellbore 104 and/or based on a condition of equipment within the wellbore 104.

The server 230 may be configured to determine one or more of the microorganism concentration based on data from the microorganism sensor 150, the type of biocide to apply, and/or one or more biocide dosages. For example, the server 230 may include the microorganism concentration calculator 202 and/or the analysis module 252 such that operation of the microorganism concentration calculator 202 and/or the analysis module 252 is performed remote from the wellbore 104, such as over the network 220. The server 230 may be located remote from the biocide application system 102 and the wellbore 104.

The client device 240 may include for example, an electronic device, such as a mobile device, such as a mobile telephone, a smart phone, a personal digital assistant (PDA), a tablet, or a laptop; or a non-mobile device, such as a desktop computer, server device, or other non-portable device. In some embodiments, the client device 240 is in operable communication with one or more of (e.g., each of) the biocide application system 102, the sensors 270, and/or the server 230 by means of the network 220.

The client device 240 may include an application for remotely monitoring and controlling one or more operations of the wellbore system 100. In some embodiments, the application of the client device 240 receives an indication of the biocide application (e.g., the type of biocide applied, at least one biocide application parameter). The client device 240 may be in operable communication with the biocide application system 102 (e.g., the biocide controller 250) and provide a real time notification of the biocide application to a wellbore operator.

In some embodiments, the client device 240 is configured to display a recommended biocide composition and/or a recommended biocide dosage to a user (e.g., a wellbore operator). The client device 240 may be configured to receive one or more inputs from the user to, for example, proceed with the recommended biocide and/or biocide application parameter. For example, the application of the client device 240 may be configured to receive one or more inputs from the user to apply a particular biocide, to apply a particular flowrate of the biocide, to apply the biocide at a particular frequency, and/or to apply the biocide for a particular duration.

The network 220 may be any type of network. For example, the network 220 may be a local network, such as a wireless or wired local network. In some examples, the network 220 may include the Internet. In some embodiments, the network 220 is a Bluetooth® network.

It should be appreciated that the environment 201 and the biocide application system 102 are only one example of an environment and a biocide application system, and that the environment 201 and the biocide application system 102 may have more or fewer components than shown, may combine additional components not depicted in the embodiment illustrated in FIG. 2, and/or may have different configurations or arrangements of components illustrated in FIG. 2. In addition, the various components illustrated in FIG. 2 may be implemented in hardware, software, or a combination of both hardware and software, including one or more signal processing and/or application specific integrated circuits. In addition, the operations of the biocide application system 102 and/or the environment as described herein may be implemented by running one or more functional modules in an information processing apparatus such as application specific chips, such as application-specific integrated circuits (ASICs), field-programmable gate arrays (FPGAs), programmable logic devices (PLDs), systems on a chip (SOCs), or other appropriate devices. These modules, combinations of these modules, and/or their combination with hardware are all included within the scope of the embodiments described herein.

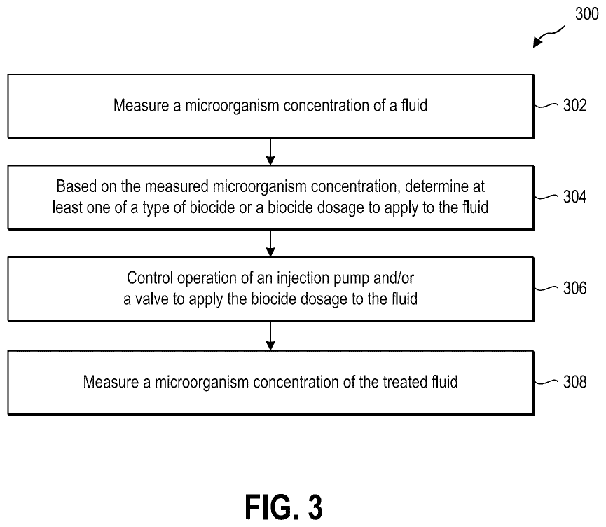

FIG. 3 is a simplified flow diagram illustrating a method 300 of controlling biocide application, according to at least one embodiment of the disclosure. The method 300 includes measuring a microorganism concentration of a fluid, as shown in act 302. Measuring the microorganism concentration of the fluid may include measuring the microorganism concentration using a microorganism sensor, as described above with reference to the microorganism sensor 150. In some embodiments, measuring the microorganism concentration includes measuring the microorganism concentration in-situ in real time. For example, the microorganism concentration may be measured at a predetermined frequency using an in-line sensor configured to autonomously collect a sample of the fluid and determine the microorganism concentration of the fluid.

Responsive to measuring the microorganism concentration of the fluid, the method 300 further includes, based on the measured microorganism concentration, determining at least one of a type of biocide to apply or a biocide dosage to apply to the fluid, as shown in act 304. In some embodiments, a biocide determination and application controller (e.g., the analysis module 252) determines at least one of the type of biocide to apply or a biocide dosage to apply, as described above.

Responsive to determining the at least one of the type of biocide or the biocide dosage, the method 300 includes controlling operation of an injection pump and/or a valve to apply the biocide dosage to the fluid to form a treated fluid, as shown in act 306. The injection pump may be controlled to apply the biocide at biocide dosage by, for example, operating the injection pump at a particular frequency (corresponding to the biocide dosage), for a duration, and/or by increasing or decreasing the speed of the injection pump. In addition, the opening and closing of the valve may be controlled to apply the biocide at the biocide dosage. In some embodiments, a valve in fluid communication with a selected type of biocide is opened and valves in fluid communication with other types of biocides are closed.

With continued reference to FIG. 3, responsive to applying the biocide dosage, the method 300 may further include measuring a microorganism concentration of the treated fluid, as shown in act 308. Measuring the microorganism concentration of the treated fluid may include measuring the microorganism concentration downstream of where the biocide was applied to the fluid. A difference between the microorganism concentration of the treated fluid measured in act 308 and the microorganism concentration of the fluid measured in act 302 may be an indication of the effectiveness of the biocide application.

Accordingly, the biocide application system 102 may be configured to measure and control the microorganism concentration of a fluid in-situ, in real time, and without operator intervention. The biocide application system 102 may reduce operating expenses of a facility that includes fluids that include microorganisms. For example, the activity of microorganisms is responsible for the corrosion of equipment, plugging of petroleum formation, and souring of the reservoir. The annual cost of biological corrosion to the oil and gas industry is estimated at $13.4 billion. To prevent this damage, bacteria populations in oil and gas fluids are commonly controlled by the application of biocides. Accurate, rapid bacteria population measurement is required to select, apply, and evaluate biocide applications. The complication, expense, and level of expertise required to measure bacteria populations means that oil field sites typically test for bacteria levels less than once a year. To ensure bacteria populations are representative of the location, water samples should be processed immediately, or at a minimum, within 12 to 24 hours of sample collection. Remote facilities can have limited sample transportation options that can preclude the timely delivery of bacteria samples to commercial laboratories, necessitating on-site bacteriological analysis.

The biocide application system 102 may be suitable for long-term operation for fluids (waters) having relatively low, medium, and high concentrations of microorganisms. The biocide application system 102 may facilitate reducing microbiologically induced corrosion (MIC) and other issues that may be caused by the presence of microorganisms. The biocide application system 102 facilitates continuous (e.g., substantially continuous, such as multiple times a day or multiple times per week) determination and control of a biocide composition and/or biocide dosage to be applied to a fluid to reduce and/or control microorganism concentrations in the fluid. The biocide application system 102 facilitates reducing the risk of MIC and oil and gas reservoir souring caused by microorganisms. For example, the biocide application system 102 facilitates pairing the output of the microorganism measurement manager 200 (e.g., the microorganism sensor 150) with the application of biocide.

The biocide application system 102 may be used to facilitate the automated control of biocide application on hundreds of offshore platforms that apply biocide to seawater before injection, in freshwater frac equipment sets, or other applications. The biocide application system 102 may also be used to determine water bacteria content and ensure proper biocide application to prevent frac formation bacteria souring. The biocide application system 102 offers onsite, accurate bacteria population data while it eliminates operator error, brings information about bacteria populations to the client device 240 (e.g., desktop), and adds the capability to automate biocide application based on need.

The online, automated bacteria population determination devices, systems, and methods described herein (e.g., the biocide application system 102) can advantageously allow for testing as often as once an hour and reporting of the results online. Prior art bacteria population measurement methods include the Bactiquant method, a manual bacteria metabolism based assay. The biocide application system 102 including the microorganism measurement manager 200 and the biocide controller 250 facilitates automatic measurement of microorganism concentration and biocide application based on the microorganism concentration without operator intervention.

The embodiments of the biocide application system 102 including the microorganism measurement manager 200 and the biocide controller 250 have been primarily described with reference to wellbore operations; the biocide application system 102 described herein may be used in applications other than the wellbore operations. In other embodiments, the biocide application system 102 according to the present disclosure may be used outside a wellbore or other downhole environment used for the exploration or production of natural resources. Accordingly, the terms “wellbore,” “borehole,” and the like should not be interpreted to limit tools, systems, assemblies, or methods of the present disclosure to any particular industry, field, or environment. In addition, the wellbore fluids may be used in cased completion wellbores and in open hole completion wellbores.

The biocide application system 102 may be used in wellbores used for hydrocarbon recovery, production of geothermal energy, injection of one or more chemicals into an earth formation and/or reservoir in the earth formation, carbon storage, or another purpose. In some embodiments, the biocide application system 102 may be used for wellbores used for carbon capture, utilization, and storage (CCUS) and/or for recovery and use of geothermal energy.

Geothermal energy is a promising source of renewable energy that captures energy from heat generated within the earth. For example, geothermal energy may be used to heat structures (e.g., buildings) and/or to generate electricity (e.g., by heating water to generate steam and drive a turbine with the steam). The biocide application system 102 described herein may be used to form wellbores used to circulate a fluid that is heated within the earth formation through which the wellbore extends. The heated fluid may be circulated to the surface where the captured heat may be recovered to heat a structure and/or generate electricity, followed by recirculation of the fluid to the earth formation to continue the cycle.

CCUS facilitates the capture, use, and/or storage of carbon (e.g., carbon dioxide), which has a goal of achieving carbon neutrality and/or net zero carbon emissions (NZE). CCUS may facilitate the capture of carbon dioxide from large point sources (e.g., power plants, refineries, cement plants, other industrial processing plants, or other industrial facilities that use fossil fuels, biomass fuels, or other fuels that generate carbon dioxide). The captured carbon dioxide may be converted into valuable products such as, for example, ethanol, sustainable aviation fuel, chemicals, and mineral aggregates. Alternatively, the carbon dioxide may be stored in geologic formations, such as in depleted hydrocarbon reservoirs. The carbon dioxide may be introduced into the earth formation through a wellbore that uses or used the biocide application system 102 during operations or formation thereof.

FIG. 4 illustrates certain components that may be included within a computer system 400, in accordance with at least one embodiment of the disclosure. One or more computer systems 400 may be used to implement the various devices, components, systems, and methods described herein. For example, one or more of the biocide application system 102, the microorganism measurement manager 200, the biocide controller 250, the server 230, and/or the client device 240 may include a computer system. In addition, the method 300 may be performed with a computer system.

The computer system 400 includes a processor 401. The processor 401 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 401 may be referred to as a central processing unit (CPU). Although just a single processor 401 is shown in the computer system 400 of FIG. 4, in an alternative configuration, a combination of processors (e.g., an ARM and DSP) could be used.

The computer system 400 also includes memory 403 in electronic communication with the processor 401. The memory 403 may be any electronic component capable of storing electronic information. For example, the memory 403 may be embodied as random-access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 405 and data 407 may be stored in the memory 403. The instructions 405 may be executable by the processor 401 to implement some or all of the functionality disclosed herein. Executing the instructions 405 may involve the use of the data 407 that is stored in the memory 403. Any of the various examples of modules and components described herein may be implemented, partially or wholly, as instructions 405 stored in memory 403 and executed by the processor 401. Any of the various examples of data described herein may be among the data 407 that is stored in memory 403 and used during execution of the instructions 405 by the processor 401.

The computer system 400 may also include one or more communication interfaces 409 for communicating with other electronic devices. The communication interface(s) 409 may be based on wired communication technology, wireless communication technology, or both. Some examples of communication interfaces 409 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

The computer system 400 may also include one or more input devices 411 and one or more output devices 413. Some examples of input devices 411 include a keyboard, mouse, microphone, remote control device, button, joystick, trackball, touchpad, and lightpen. Some examples of output devices 413 include a speaker and a printer. One specific type of output device that is typically included in a computer system 400 is a display device 415. Display devices 415 used with embodiments disclosed herein may utilize any suitable image projection technology, such as liquid crystal display (LCD), light-emitting diode (LED), gas plasma, electroluminescence, or the like. A display controller 417 may also be provided, for converting data 407 stored in the memory 403 into text, graphics, and/or moving images (as appropriate) shown on the display device 415.

The various components of the computer system 400 may be coupled together by one or more buses, which may include a power bus, a control signal bus, a status signal bus, a data bus, etc. For the sake of clarity, the various buses are illustrated in FIG. 4 as a bus system 419.

The techniques described herein may be implemented in hardware, software, firmware, or any combination thereof, unless specifically described as being implemented in a specific manner. Any features described as modules, components, or the like may also be implemented together in an integrated logic device or separately as discrete but interoperable logic devices. If implemented in software, the techniques may be realized at least in part by a non-transitory processor-readable (computer-readable) storage medium comprising (having) instructions thereon that, when executed by at least one processor, perform one or more of the methods described herein. The instructions may be organized into routines, programs, objects, components, data structures, etc., which may perform particular tasks and/or implement particular data types, and which may be combined or distributed as desired in various embodiments.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and/or within less than 0.01% of the stated amount. As another example, in certain embodiments, the terms “generally parallel” and “substantially parallel” or “generally perpendicular” and “substantially perpendicular” refer to a value, amount, or characteristic that departs from exactly parallel or perpendicular, respectively, by less than or equal to 15 degrees, 10 degrees, 5 degrees, 3 degrees, 1 degree, or 0.1 degree. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

Although a few embodiments of the disclosure have been described in detail above, those of ordinary skill in the art will readily appreciate that many modifications are possible without materially departing from the teachings of this disclosure. Accordingly, such modifications are intended to be included within the scope of this disclosure as defined in the claims. It is also contemplated that various combinations or sub-combinations of the specific features and aspects of the embodiments described may be made and still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above.

What is claimed is:

1. A system for measuring a microorganism concentration and applying a biocide, the system comprising:
an injection pump in fluid communication with at least one biocide tank and a fluid conduit in fluid communication with a wellbore;
a microorganism sensor in fluid communication with the fluid conduit and configured to determine a microorganism concentration of fluid in the fluid conduit; and
a biocide controller in operable communication with the injection pump and the microorganism sensor, the biocide controller comprising:
at least one processor; and
at least one non-transitory computer-readable storage medium having instructions thereon that, when executed by the at least one processor, cause the biocide controller to:
receive, from the microorganism sensor, the microorganism concentration of the fluid in the fluid conduit;
based on the microorganism concentration of the fluid in the fluid conduit, determine a biocide dosage to apply to the fluid; and
control an operation of the injection pump to apply the biocide dosage to the fluid.

2. The system of claim 1, wherein the microorganism sensor is configured to determine the microorganism concentration of the fluid in the fluid conduit on at predetermined schedule.

3. The system of claim 1, wherein the instructions, when executed by the at least one processor, cause the biocide controller to:
determine a concentration of the biocide to apply based on the microorganism concentration of the fluid; and
control the operation of the injection pump to apply the concentration of the biocide to the fluid.

4. The system of claim 1, wherein the instructions, when executed by the at least one processor, cause the biocide controller to determine a frequency and duration of application of the biocide based on the microorganism concentration of the fluid.

5. The system of claim 4, wherein the instructions, when executed by the at least one processor, cause the biocide controller to cause the injection pump to apply the biocide to the fluid at the frequency and duration.

6. The system of claim 1, further comprising at least one sensor configured to determine one or more conditions within the wellbore or conditions of equipment within the wellbore.

7. The system of claim 6, wherein the instructions, when executed by the at least one processor, cause the biocide controller to determine the biocide dosage based on an output from the at least one sensor.

8. The system of claim 1, wherein the instructions, when executed by the at least one processor, cause the biocide controller to determine the biocide dosage based, at least in part, on a cost of the biocide.

9. The system of claim 1, wherein the at least one biocide tank includes a first biocide tank including a first biocide and a second biocide tank including a second biocide.

10. The system of claim 9, wherein the instructions, when executed by the at least one processor, cause the biocide controller to determine a type of biocide to apply based on the microorganism concentration of the fluid.

11. The system of claim 10, wherein the instructions, when executed by the at least one processor, cause the biocide controller to open a valve in fluid communication with a selected type of biocide.

12. The system of claim 1, wherein the fluid includes water.

13. A method of autonomously treating a fluid with biocide, the method comprising:
measuring a microorganism concentration with a microorganism sensor in fluid communication with a fluid conduit configured to provide a fluid to a wellbore;
receiving, with an analysis module, the microorganism concentration from the microorganism sensor;
using the analysis module, determining a biocide dosage to apply to the fluid to reduce the microorganism concentration in the fluid based on the measured microorganism concentration in the fluid; and
based on the determined biocide dosage, controlling an operation of an injection pump with a biocide application controller in operable communication with the analysis module.

14. The method of claim 13, wherein controlling an operation of an injection pump with a biocide application controller includes controlling one or more of a flowrate, a duration, or a frequency of application of the biocide with the injection pump.

15. The method of claim 13, wherein measuring a microorganism concentration includes measuring the microorganism concentration at a frequency of at least two times per day.

16. The method of claim 13, wherein determining a biocide dosage to apply to the fluid to reduce the microorganism concentration in the fluid based on the measured microorganism concentration in the fluid includes determining biocide composition to apply based on the measured microorganism concentration.

17. The method of claim 13, wherein controlling an operation of an injection pump with a biocide application controller includes controlling one or more of a speed of the injection pump, a frequency of operation of the injection pump, or a duration of operation of the injection pump.

18. The method of claim 13, wherein controlling an operation of an injection pump with a biocide application controller includes controlling the injection pump based on a composition of a produced fluid.

19. The method of claim 13, wherein controlling an operation of an injection pump with a biocide application controller includes controlling the injection pump based on a cost of the biocide.

20. A biocide application system, comprising:
a microorganism sensor configured to determine a microorganism concentration within a fluid; and
a biocide controller comprising:
at least one processor; and
at least one non-transitory computer-readable storage medium having instructions thereon that, when executed by the at least one processor, cause the biocide controller to:
receive, from the microorganism sensor, the microorganism concentration;
based on the microorganism concentration, determine at least one of a biocide flowrate, a biocide concentration, a biocide application duration, or a biocide application frequency to apply to the fluid; and
cause a biocide injection pump to provide biocide to the fluid based on the at least one of the biocide flowrate, the biocide concentration, the biocide application duration, or the biocide application frequency.
